SEQUENCE LISTING (133 sequences)

SEQ ID NO: 1 - protein, 8 aa, Homo sapiens
  MISC_FEATURE (4)..(4): Xaa is hydroxyproline
EGPXGPQG

SEQ ID NO: 2 - protein, 7 aa, Homo sapiens
  MISC_FEATURE (3)..(3): Xaa is hydroxyproline
GPXGPQG

SEQ ID NO: 3 - protein, 5 aa, Homo sapiens
TIINE

SEQ ID NO: 4 - protein, 6 aa, Homo sapiens
GPPGQG

SEQ ID NO: 5 - protein, 9 aa, Homo sapiens
GEPGDDAPS

SEQ ID NO: 6 - protein, 20 aa, Bos taurus
PGDDGEAGKPGKSGERGPPG

SEQ ID NO: 7 - protein, 20 aa, Homo sapiens
PGDDGEAGKPGKAGERGPPG

SEQ ID NO: 8 - protein, 7 aa, Homo sapiens
GEAGKPG

SEQ ID NO: 9 - protein, 7 aa, Homo sapiens
GPPGPQG

SEQ ID NO: 10 - protein, 10 aa, Homo sapiens
GEPGDDGPSG

SEQ ID NO: 11 - protein, 13 aa, Homo sapiens
APGEDGRPGPPGP

SEQ ID NO: 12 - protein, 16 aa, Homo sapiens
GKVGPSGAPGEDGRPG

SEQ ID NO: 13 - protein, 9 aa, Homo sapiens
AEGPPGPQG

SEQ ID NO: 14 - protein, 10 aa, Homo sapiens
GPPGPQGLAG

SEQ ID NO: 15 - protein, 9 aa, Homo sapiens
GEPGDDGPS

SEQ ID NO: 16 - protein, 16 aa, Homo sapiens
GEPGDDGPSGAEGPPG

SEQ ID NO: 17 - protein, 21 aa, Homo sapiens
EKGEPGDDAPSGAEGPPGPQG

SEQ ID NO: 18 - protein, 21 aa, Homo sapiens
GPPGPPGKPGDDGEAGKPGKA

SEQ ID NO: 19 - protein, 21 aa, Homo sapiens
GPPGPRGRSGETGPAGPPGNP

SEQ ID NO: 20 - protein, 22 aa, Homo sapiens
GAPGPQGFQGNPGEPGEPGVSY

SEQ ID NO: 21 - protein, 20 aa, Homo sapiens
GEPGDDAGPSGAEGPPGPQG

SEQ ID NO: 22 - protein, 9 aa, Homo sapiens
HRGYPGLDG

SEQ ID NO: 23 - protein, 23 aa, Homo sapiens
CGKVGPSGAPGEDGRPGPPGPQY

SEQ ID NO: 24 - protein, 13 aa, Homo sapiens
APGEDGRPGPPGP

SEQ ID NO: 25 - protein, 4 aa, Homo sapiens
GQPG

SEQ ID NO: 26 - protein, 11 aa, Homo sapiens
CGGEGPPGPQG

SEQ ID NO: 27 - protein, 19 aa, Homo sapiens
GAEGPPGPQGLAGQRGIVG

SEQ ID NO: 28 - protein, 19 aa, Homo sapiens
GAPGTPGPQGIAGQRGVVG

SEQ ID NO: 29 - protein, 19 aa, Homo sapiens
GPPGTPGPQGLLGAPGILG

SEQ ID NO: 30 - protein, 19 aa, Homo sapiens
GPPGAPGPLGIAGITGARG

SEQ ID NO: 31 - protein, 12 aa, Homo sapiens
CGGEGPPGPQGL

SEQ ID NO: 32 - protein, 13 aa, Homo sapiens
CGGEGPPGPQGLA

SEQ ID NO: 33 - protein, 10 aa, Homo sapiens
CGGEGPPGPQ

SEQ ID NO: 34 - protein, 9 aa, Homo sapiens
CGGEGPPGP

SEQ ID NO: 35 - protein, 8 aa, Homo sapiens
CGPPGPQG

SEQ ID NO: 36 - protein, 30 aa, Homo sapiens
LQGPAGPPGEKGEPGDDGPSGAEGPPGPQG

SEQ ID NO: 37 - protein, 5 aa, Homo sapiens
PGPQG

SEQ ID NO: 38 - protein, 6 aa, Homo sapiens
PPGPQG

SEQ ID NO: 39 - protein, 31 aa, Homo sapiens
VLQGPAGPPGEKGEPGDDGPSGAEGPPGPQG

SEQ ID NO: 40 - protein, 47 aa, Homo sapiens
KGARGDSGPPGRAGEPGLQGPAGPPGEKGEPGDDGPSGAEGPPGPQG

SEQ ID NO: 41 - protein, 45 aa, Homo sapiens
ARGDSGPPGRAGEPGLQGPAGPPGEKGEPGDDGPSGAEGPPGPQG

SEQ ID NO: 42 - protein, 28 aa, Homo sapiens
GPAGPPGEKGEPGDDGPSGAEGPPGPQG

SEQ ID NO: 43 - protein, 145 aa, Homo sapiens
GPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLGLPGKDGETGAEGPPPA

SEQ ID NO: 44 - protein, 347 aa, Homo sapiens
MPGERGAAGIAGPKGDRGDVGEKGPEGAPGKDGGRGLTGPIGPPGPAGANGEKGEVGPPGPAGSAGARGAPGERGETGPPGTSGIAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGQQGAPGASGDRGPPGPVGPPGLTGPAGEPGREGSPGADGPPGRDGAAGVKGDRGETGAVGAPGAPGPPGSPGPAGPTGKQGDRGEAGAQGPM

SEQ ID NO: 45 - protein, 6 aa, Homo sapiens
EKGPDP

SEQ ID NO: 46 - protein, 7 aa, Homo sapiens
  MISC_FEATURE (4)..(5): isomerised peptide bond
GDIKDIV

SEQ ID NO: 47 - protein, 5 aa, Homo sapiens
  MISC_FEATURE (4)..(5): Isomerised peptide bond
EKGPD

SEQ ID NO: 48 - protein, 7 aa, Homo sapiens
GDIKDIV

SEQ ID NO: 49 - protein, 5 aa, Homo sapiens
EKGPD

SEQ ID NO: 50 - protein, 5 aa, Homo sapiens
PGVKG

SEQ ID NO: 51 - protein, 6 aa, Homo sapiens
PGPKGE

SEQ ID NO: 52 - protein, 6 aa, Homo sapiens
GQKGEP

SEQ ID NO: 53 - protein, 13 aa, Homo sapiens
  MISC_FEATURE (6)..(6): unspecified amino acid
GFQGLXGXXGXXG

SEQ ID NO: 54 - protein, 13 aa, Homo sapiens
  MISC_FEATURE (6)..(6): unspecified amino acid
GLQGLXGXXGXSG

SEQ ID NO: 55 - protein, 6 aa, Homo sapiens
GQPGPA

SEQ ID NO: 56 - protein, 6 aa, Homo sapiens
EPGGVG

SEQ ID NO: 57 - protein, 6 aa, Homo sapiens
DQGVPG

SEQ ID NO: 58 - protein, 6 aa, Homo sapiens
PKGARG

SEQ ID NO: 59 - protein, 6 aa, Homo sapiens
REGSPG

SEQ ID NO: 60 - protein, 5 aa, Homo sapiens
  MISC_FEATURE (3)..(4): isomerised peptide bond
GADGP

SEQ ID NO: 61 - protein, 13 aa, Homo sapiens
  MISC_FEATURE (3)..(3): hydroxyproline
GSPGADGPPGRKK

SEQ ID NO: 62 - protein, 16 aa, Homo sapiens
AQGPPGATGFPGAAGR

SEQ ID NO: 63 - protein, 11 aa, Homo sapiens
ASGDRGPPGPV

SEQ ID NO: 64 - protein, 15 aa, Homo sapiens
ASGDRGPPGPVGPPG

SEQ ID NO: 65 - protein, 15 aa, Homo sapiens
GANGEKGEVGPPGPA

SEQ ID NO: 66 - protein, 15 aa, Homo sapiens
GAPGEDGRPGPPGPQ

SEQ ID NO: 67 - protein, 18 aa, Homo sapiens
GARGAPGERGETGPPGPA

SEQ ID NO: 68 - protein, 9 aa, Homo sapiens
GDRGPPGPV

SEQ ID NO: 69 - protein, 7 aa, Homo sapiens
GERGFPG

SEQ ID NO: 70 - protein, 9 aa, Homo sapiens
GERGFPGER

SEQ ID NO: 71 - protein, 15 aa, Homo sapiens
GESGSPGENGSPGPM

SEQ ID NO: 72 - protein, 16 aa, Homo sapiens
GLPGPPGPPGEGGKPG

SEQ ID NO: 73 - protein, 9 aa, Homo sapiens
GPIGPPGPA

SEQ ID NO: 74 - protein, 19 aa, Homo sapiens
GPPGPPGKPGDDGEAGKPG

SEQ ID NO: 75 - protein, 6 aa, Homo sapiens
GPPGPV

SEQ ID NO: 76 - protein, 9 aa, Homo sapiens
GPPGPVGPA

SEQ ID NO: 77 - protein, 15 aa, Homo sapiens
LPGPPGPPGEGGKPG

SEQ ID NO: 78 - protein, 14 aa, Homo sapiens
NPGPPGPPGPPGPG

SEQ ID NO: 79 - protein, 5 aa, Homo sapiens
PIGPP

SEQ ID NO: 80 - protein, 20 aa, Homo sapiens
REGSPGADGPPGRDGAAGVK

SEQ ID NO: 81 - protein, 14 aa, Homo sapiens
SNGNPGPPGPPGPS

SEQ ID NO: 82 - protein, 1060 aa, Homo sapiens
QMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRA

SEQ ID NO: 83 - protein, 23 aa, Homo sapiens
AAGARGNDGQPGPAGPPGPVGPA

SEQ ID NO: 84 - protein, 18 aa, Homo sapiens
AQGPRGEPGTPGSPGPAG

SEQ ID NO: 85 - protein, 18 aa, Homo sapiens
ARGAPGERGETGPPGPAG

SEQ ID NO: 86 - protein, 11 aa, Homo sapiens
ASGDRGPPGPV

SEQ ID NO: 87 - protein, 12 aa, Homo sapiens
ASGDRGPPGPVG

SEQ ID NO: 88 - protein, 9 aa, Homo sapiens
ATGPLGPKG

SEQ ID NO: 89 - protein, 12 aa, Homo sapiens
DRGPPGPVGPPG

SEQ ID NO: 90 - protein, 30 aa, Homo sapiens
ERGAPGNRGFPGQDGLAGPKGAPGERGPSG

SEQ ID NO: 91 - protein, 35 aa, Homo sapiens
FQGLPGPPGPPGEGGKPGDQGVPGEAGAPGLVGPR

SEQ ID NO: 92 - protein, 36 aa, Homo sapiens
FQGLPGPPGPPGEGGKPGDQGVPGEAGAPGLVGPRG

SEQ ID NO: 93 - protein, 15 aa, Homo sapiens
FTGLQGLPGPPGPSG

SEQ ID NO: 94 - protein, 39 aa, Homo sapiens
FTGLQGLPGPPGPSGDQGASGPAGPSGPRGPPGPVGPSG

SEQ ID NO: 95 - protein, 15 aa, Homo sapiens
GANGEKGEVGPPGPA

SEQ ID NO: 96 - protein, 15 aa, Homo sapiens
GAPGEDGRPGPPGPQ

SEQ ID NO: 97 - protein, 16 aa, Homo sapiens
GAPGEDGRPGPPGPQG

SEQ ID NO: 98 - protein, 15 aa, Homo sapiens
GAPGERGETGPPGPA

SEQ ID NO: 99 - protein, 15 aa, Homo sapiens
GESGSPGENGSPGPM

SEQ ID NO: 100 - protein, 9 aa, Homo sapiens
GPIGPPGPA

SEQ ID NO: 101 - protein, 10 aa, Homo sapiens
GPPGPVGPPG

SEQ ID NO: 102 - protein, 16 aa, Homo sapiens
GPRGPPGPAGAPGPQG

SEQ ID NO: 103 - protein, 26 aa, Homo sapiens
GVMQGPMGPMGPRGPPGPAGAPGPQG

SEQ ID NO: 104 - protein, 25 aa, Homo sapiens
IVGLPGQRGERGFPGLPGPSGEPGK

SEQ ID NO: 105 - protein, 21 aa, Homo sapiens
KQGDRGEAGAQGPMGPSGPAG

SEQ ID NO: 106 - protein, 21 aa, Homo sapiens
KVGPSGAPGEDGRPGPPGPQG

SEQ ID NO: 107 - protein, 21 aa, Homo sapiens
LPGKDGETGAAGPPGPAGPAG

SEQ ID NO: 108 - protein, 33 aa, Homo sapiens
LPGKDGETGAAGPPGPAGPAGERGEQGAPGPSG

SEQ ID NO: 109 - protein, 36 aa, Homo sapiens
LQGLPGPPGPSGDQGASGPAGPSGPRGPPGPVGPSG

SEQ ID NO: 110 - protein, 17 aa, Homo sapiens
LTGPAGEPGREGSPGAD

SEQ ID NO: 111 - protein, 27 aa, Homo sapiens
LTGPAGEPGREGSPGADGPPGRDGAAG

SEQ ID NO: 112 - protein, 12 aa, Homo sapiens
LTGPIGPPGPAG

SEQ ID NO: 113 - protein, 33 aa, Homo sapiens
LTGRPGDAGPQGKVGPSGAPGEDGRPGPPGPQG

SEQ ID NO: 114 - protein, 23 aa, Homo sapiens
QGPMGPMGPRGPPGPAGAPGPQG

SEQ ID NO: 115 - protein, 31 aa, Homo sapiens
QGPRGLPGTPGTDGPKGASGPAGPPGAQGPP

SEQ ID NO: 116 - protein, 28 aa, Homo sapiens
RSGETGPAGPPGNPGPPGPPGPPGPGID

SEQ ID NO: 117 - protein, 21 aa, Homo sapiens
RVGPPGSNGNPGPPGPPGPSG

SEQ ID NO: 118 - protein, 14 aa, Homo sapiens
SNGNPGPPGPPGPS

SEQ ID NO: 119 - protein, 9 aa, Homo sapiens
SPGPMGPRG

SEQ ID NO: 120 - protein, 21 aa, Homo sapiens
VKGESGSPGENGSPGPMGPRG

SEQ ID NO: 121 - protein, 11 aa, Homo sapiens
REGSPGADGPP

SEQ ID NO: 122 - protein, 22 aa, Homo sapiens
LTGPAGEPGREGSPGADGPPGR

SEQ ID NO: 123 - protein, 16 aa, Homo sapiens
GSPGADGPPGRDGAAG

SEQ ID NO: 124 - protein, 10 aa, Homo sapiens
LTGPAGGGGC

SEQ ID NO: 125 - protein, 10 aa, Homo sapiens
LTGPAGEPGK

SEQ ID NO: 126 - protein, 11 aa, Artificial
  Random sequence
GLTGPAGEPGK

SEQ ID NO: 127 - protein, 10 aa, Artificial
  random sequence
KGATGPLGPK

SEQ ID NO: 128 - protein, 10 aa, Homo sapiens
  MISC_FEATURE (2)..(2): Hydroxyproline
SPGADGPPGR

SEQ ID NO: 129 - protein, 11 aa, Homo sapiens
  MISC_FEATURE (3)..(3): Hydroxyproline
CSPGADGPPGR

SEQ ID NO: 130 - protein, 11 aa, Homo sapiens
  MISC_FEATURE (3)..(3): Hydroxyproline
GSPGADGPPGR

SEQ ID NO: 131 - protein, 29 aa, Homo sapiens
  MISC_FEATURE (17)..(18): Isomerised peptide bond
LTGPAGEPGREGSPGADGPPGRDGAAGVK

SEQ ID NO: 132 - protein, 6 aa, Homo sapiens
EAGKPG

SEQ ID NO: 133 - protein, 11 aa, Homo sapiens
REGSPGGADAP

(19) United States
(12) Patent Application Publication
(10) Pub. No.: US 2011/0256639 A1
(43) Pub. Date: Oct. 20, 2011

(54) ASSESSMENT OF PROTEIN DEGRADATION BY MEASUREMENT OF ISOMERISED NEO-EPITOPE CONTAINING FRAGMENTS

(75) Inventors: Diana J. Leeming, Copenhagen (DK); Inger Byrjalsen, Hiersholm (DK); Per Qvist, Klampenborg (DK); Morten A. Karsdal, Copenhagen (DK)

(73) Assignee: Nordic Biosciences A/S, Herlev (DK)

(21) Appl. No.: 13/128,935

(22) PCT Filed: Nov. 11, 2009
(86) PCT No.: PCT/EP09/64995

§ 371 Date: Jun. 23, 2011

(30) Foreign Application Priority Data
Nov. 13, 2008 (GB) 0820784.7

(51) Int. Cl. G01N 33/566

(52) U.S. Cl. 436/501

(57) ABSTRACT

A method of immunoassay for fragments of a protein such as type II collagen in a biological sample detects fragments having a first epitope containing an isomerised amino acid residue and a second epitope generated by cleavage of the protein by the use of respective antibodies binding each of the two epitopes.

The invention relates to the measurement of amounts of protein fragments in body samples where the fragments combine a neo-epitope for antibody binding generated by cleavage of the relevant protein by a protease in the body and an epitope containing an isomerisation, which may act as an age marker for the protein. It is an unexpected discovery that certain peptide fragments naturally occurring in samples taken from the human or animal body contain two such epitopes by means of which they can be quantitated in an immunological assay procedure.

Osteoarthritis is one of the leading causes of disability in the world, with more than 10% of the elderly population having symptomatic disease (Woolf & Pfleger, 2003). The incidence increases with age, and by age 65, 80% has radiographic evidence of OA (Lawrence et al., 1998). Therefore, osteoarthritis is both prevalent and a serious burden to the patient and the society. However, at present there is little to offer the affected individuals for prevention of the disease or treatment in the early stages. For many patients, hip or knee replacement is eventually the only treatment option.

Although the pathogenicity of osteoarthritis is not fully understood at present, it is evident that a central hallmark in this slow, chronic disease is progressive destruction of the articular joints, which consists of bone, cartilage and the synovium. In particular the cartilage has attracted much attention, and the different grades and stages of OA cartilage histopathology have recently been detailed described by a working group under OARSI (Pritzker et al., 2006). This system encompasses 7 grades (or severity levels) with involvement of the deeper cartilage layers in more advanced OA disease, and when combined with the extent of cartilage involvement expressed in 5 stages this leads to a semi-quantitative scoring system of 0 to 24.

However, the lack of sensitive, specific and fast analytical techniques to assess important metabolic processes in articular cartilage and their effects on the structure of the tissue is a major barrier to effective drug development in OA.

In particular, major efforts have been allocated to the development of new and better biochemical markers of cartilage turnover.

Cartilage, including articular cartilage, is for the most part composed of collagen type II (60%-70% of dry weight) and proteoglycans (10% of dry weight). Cartilage degradation is mainly mediated by the MMPs and the closely related ADAM-TS (a disintegrin and metalloproteinase with thrombospondin motifs), but collagen type II is most sensitive to MMP activity (Dean et al., 1989; Reboul et al., 1996; Hui et al., 2003). The action of these proteases results in the release of various extracellular fragments which could be candidates as biomarkers of cartilage degradation.

Proteolytic cleavage of type II collagen has been reported in numerous studies, and several different degradation fragments of collagen type II have been indicated as useful for monitoring degenerative diseases of the cartilage (Schaller et al., 2005; Sumer et al., 2006; Birmingham et al., 2006).

A fragment of the C-telopeptide of type II collagen, i.e. CTX-II, is generated by MMP-activity (Christgau et al., 2001; Mouritzen et al., 2003) and measurement of CTX-II has been reported for monitoring degradation of type II collagen in experimental setups assessing cartilage degradation (Schaller et al., 2005) as well as in humans (Reijman et al., 2004).

However, the first report of using antibodies for detection of collagen type II fragments came from Billinghurst and co-workers (Billinghurst et al., 1997), who described detection of an amino-terminal neoepitope on the shorter fragment of type II collagen after cleavage by collagenase.

More well-described in the literature is the C2C neoepitope at the C-terminus of the length fragment (Fraser et al., 2003; Poole et al., 2004). This test is dependant of the binding of a monoclonal antibody to the amino acid sequence EGPP(OH)GPQG SEQ ID NO: 1 (Poole et al., 2004).

Other tests for collagen type II fragments include the C1,2C, which, however, is based on the amino acid sequence GPP(OH)GPQG SEQ ID NO:2 found in both type I and type II (Billinghurst et al., 1997). Also, another fragment generated by the action of collagenase is the TIINE SEQ ID NO:3 fragment (Otterness et al., 1997) and can be detected using monoclonal antibody 9A4 recognising the neoepitope Gly-Pro-Pro-Gly-Pro-Gln-Gly-COOH SEQ ID NO:4. Combined with monoclonal antibody 5109 (Downs et al., 2001) as a capture antibody, the sandwich test is claimed to be specific for type II collagen fragments.

Monoclonal antibody 5109 binds to the internal amino acid sequence GEPGDDAPS SEQ ID NO:5 and the sandwich test detects collagen fragments of variable length at the N-terminus of the binding sequence of monoclonal antibody 5109.

Cathepsin K cleavage of type II collagen has been reported in a few studies. The first study to associate cathepsin K with cleavage of type II collagen was reported by Kafienah et al. (1998). Kafienah and coworkers described helical cleavage site(s) of collagen type II by Cathepsin K, and provided the amino acid sequence for one cleavage site, i.e.

SEQ ID NO: 6
PGDDGEAGKPG [symbol] KSGERGPPG (bovine sequence)

Ten years after, Dejica et al. (2008) reported the development of an enzyme-linked immunosorbent assay based on polyclonal antibodies against the C-terminal neoepitope (C2K) of the cathepsin K cleavage site reported by Kefienah, i.e.

| | |
|---|---|
| PGDDGEAGKPG [symbol] KAGERGPPG | SEQ ID NO: 7 |

As the seven C-terminal amino acids of this epitope, i.e. GEAGKPG SEQ ID NO: 8 can be found in type I collagen as well, this test will not distinguish type I and type II collagen fragments generated by cathepsin K activity. In contrast, according to the present invention such sequences are de-selected to increase specificity for type II collagen fragments.

Zhen (2008) and co-workers discloses identification and characterization of proteolytic peptide products of human articular cartilage, including type II collagen fragments.

U.S. Pat. No. 6,642,007 (Saltarelli) disclose methods for monitoring urine for type II collagen fragment using a combination of a capture antibody and a detection antibody, such that type II collagen is distinguished from other collagen fragments.

U.S. Pat. No. 6,030,792 (Otterness) discloses antibodies for detecting collagen type II fragments resulting from collagenase cleavage. In particular, the following sequences are disclosed;

| | |
|---|---|
| GPPGPQG | SEQ ID NO: 9 |
| GEPGDDGPSG | SEQ ID NO: 10 |
| APGEDGRPGPPGP | SEQ ID NO: 11 |
| GKVGPSGAPGEDGRPG | SEQ ID NO: 12 |
| AEGPPGPQG | SEQ ID NO: 13 |
| GPPGPQGLAG | SEQ ID NO: 14 |
| GEPGDDGPS | SEQ ID NO: 15 |
| GEPGDDGPSGAEGPPG | SEQ ID NO: 16 |
| EKGEPGDDAPSGAEGPPGPQG | SEQ ID NO: 17 |
| GPPGPPGKPGDDGEAGKPGKA | SEQ ID NO: 18 |
| GPPGPRGRSGETGPAGPPGNP | SEQ ID NO: 19 |
| GAPGPQGFQGNPGEPGEPGVSY | SEQ ID NO: 20 |
| GEPGDDAGPSGAEGPPGPQG | SEQ ID NO: 21 |

A series of patents (U.S. Pat. Nos. 6,602,980; 6,566,492; 6,348,320; 6,255,056; 6,153,732; 6,143,511; 6,100,379; 5,919,634; 5,702,909; 5,688,652; 5,641,837; 5,641,687; 5,532,169; and 5,455,179) (Eyre) relates to peptides and methods for cartilage resorption assays employing antibodies binding to epitopes in the telopeptides of type II collagen.

U.S. Pat. No. 5,283,197 (Robins) describes methods of detecting collagen fragments cross-linked with lysyl pyridinoline or hydroxylysyl pyridinoline.

U.S. Pat. No. 7,410,770 (Reginster) disclosed methods for detection of collagenase-generated fragments of collagen type II using antibody binding to epitope in the amino acid sequence HRGYPGLDG SEQ ID NO:22 located in the helical region of collagen type II.

U.S. Pat. No. 6,132,976 (Poole) discloses methods for detecting cartilage degradation using antibodies which does not bind to unwound (native) type II collagen fragments but only to fragments being generated by collagenase cleavage. In particular the following amino acid sequences originating from type II collagen are included;

| | |
|---|---|
| CGKVGPSGAPGEDGRPGPPGPQY | SEQ ID NO: 23 |
| APGEDGRPGPPGP | SEQ ID NO: 24 |
| GQPG | SEQ ID NO: 25 |
| GPPGPQG | SEQ ID NO: 9 |
| CGGEGPPGPQG | SEQ ID NO: 26 |
| GAEGPPGPQGLAGQRGIVG | SEQ ID NO: 27 |
| GAPGTPGPQGIAGQRGVVG | SEQ ID NO: 28 |
| GPPGTPGPQGLLGAPGILG | SEQ ID NO: 29 |
| GPPGAPGPLGIAGITGARG | SEQ ID NO: 30 |
| CGGEGPPGPQGL | SEQ ID NO: 31 |
| CGGEGPPGPQGLA | SEQ ID NO: 32 |
| CGGEGPPGPQ | SEQ ID NO: 33 |
| CGGEGPPGP | SEQ ID NO: 34 |
| CGPPGPQG | SEQ ID NO: 35 |

U.S. Pat. No. 7,115,378 (Welsch) describes the use of mass spectrometry for identifying and quantifying peptides resulting from enzyme cleavage of collagen type II. The technique is used for identification and quantification of the peptides in a biological sample to assess activity of proteolytic enzymes in osteoarthritis and rheumatoid arthritis. In particular, the following sequences originating from humans are disclosed;

SEQ ID NO: 9
GPPGPQG
SEQ ID NO: 36
LQGPAGPPGEKGEPGDDGPSGAEGPPGPQG
PQG
SEQ ID NO: 37
PGPQG
SEQ ID NO: 38
PPGPQG
SEQ ID NO: 39
VLQGPAGPPGEKGEPGDDGPSGAEGPPGPQG
SEQ ID NO: 40
KGARGDSGPPGRAGEPGLQGPAGPPGEKGEPGDDGPSGAEGPPGPQG
SEQ ID NO: 41
ARGDSGPPGRAGEPGLQGPAGPPGEKGEPGDDGPSGAEGPPGPQG
SEQ ID NO: 42
GPAGPPGEKGEPGDDGPSGAEGPPGPQG
SEQ ID NO: 43
GPIGPPGERGAPGNRGFPGQDGLAGPKGAPGERGPSGLAGPKGANGDPGR
PGEPGLPGARGLTGRPGDAGPQGKVGPSGAPGEDGRPGPPGPQGARGQPG
VMGFPGPKGANGEPGKAGEKGLPGAPGLGLPGKDGETGAEGPPP
A . . .

U.S. Pat. No. 6,706,490 (Cook) describes the detection of antibodies to collagen using CB peptides, in particular CB10, of mammalian type II collagen. Cyanogen bromide cleaves the carboxyl terminal of methionine residues thereby producing the CB peptides. The CB10 peptide has the sequence:

SEQ ID NO: 44
MPGERGAAGIAGPKGDRGDVGEKGPEGAPGKDGGRGLTGPIGPPGPAGAN
GEKGEVGPPGPAGSAGARGAPGERGETGPPGTSGIAGPPGADGQPGAKGE
QGEAGQKGDAGAPGPQGPSGAPGPQGPTGVTGPKGARGAQGPPGATGFPG
AAGRVGPPGSNGNPGPPGPPGPSGKDGPKGARGDSGPPGRAGEPGLQGPA
GPPGEKGEPGDDGPSGAEGPPGPQGLAGQRGIVGLPGQRGERGFPGLPGP
SGEPGQQGAPGASGDRGPPGPVGPPGLTGPAGEPGREGSPGA
DGPPGRDGAAGVKGDRGETGAVGAPGAPGPPGSPGPAGPTGKQG DRGEA
GAQGPM

U.S. Pat. No. 7,195,883 (Rosenquist) disclose sandwich immunoassays in which a single antibody specific for the amino acid sequence EKGPDP SEQ ID NO:45 is used to detect telopeptide fragments of type II collagen.

U.S. Pat. Nos. 6,420,125 and 6,107,047 (Fledelius) disclose methods of measuring the rate of degradation of collagen using antibodies binding to an amino acid sequence of type II collagen containing an isoaspartic acid residue. Also, the use of synthetic peptides having an amino acid sequence of type II collagen that contains an isoaspartic acid residue is described. In particular, the following sequences from type II collagen is described;

| | |
|---|---|
| GDIK*DIV | SEQ ID NO: 46 |
| EKGP*D, | SEQ ID NO: 47 |

where (*) denotes an isomerised peptide bond.

U.S. Pat. No. 6,300,083 (Fledelius) describes the determination of the amount of a D-amino acid containing fragment of the protein in a body fluid using an antibody capable of discriminating between the D-amino acid containing fragment and its L-amino acid containing analogue. In particular, the application includes the following peptide sequences from type II collagen;

| | |
|---|---|
| GDIKDIV | SEQ ID NO: 48 |
| EKGPD | SEQ ID NO: 49 |

U.S. Pat. Nos. 6,372,442 and 6,210,902 (Bonde) describe methods of characterizing the degradation of type II collagen. At least two distinct immunological assays should be used, each using a different immunological binding partner, and a numerical index is formed representing the difference in the results of the assays. In particular, the following type II collagen sequences are disclosed;

| | |
|---|---|
| EKGPDP | SEQ ID NO: 45 |
| EKGPD | SEQ ID NO: 49 |
| GVK | |
| PGVKG | SEQ ID NO: 50 |
| PGPKGE | SEQ ID NO: 51 |
| GQKGEP | SEQ ID NO: 52 |
| GDIKDIV | SEQ ID NO: 48 |

U.S. Pat. Nos. 6,355,442, 6,342,361, 6,323,314 and 6,110,689 (Qvist) describe the use of antibodies recognizing synthetic peptides for detection of collagen fragments. In particular, the following amino acid sequences are included;

| | |
|---|---|
| PGPKGE | SEQ ID NO: 51 |
| GQKGEP | SEQ ID NO: 52 |
| GDIKDIV | SEQ ID NO: 48 |
| EKGPD | SEQ ID NO: 49 |
| GVK | |
| PGVKG | SEQ ID NO: 50 |

U.S. Pat. No. 6,010,863 (Te Koppele) discloses the use of a sandwich immunoassay for the detection of collagen degradation using a first antibody directed at an epitope present on a collagen molecule at a distance of up to 165 amino acids from a collagen telopeptide crosslink site, and a second antibody directed at another epitope of the crosslinked collagen molecule.

U.S. Pat. No. 5,541,295 (Barrach) discloses monoclonal antibodies which bind specifically to Type II collagen, but not to its peptides, or vice versa. In particular, the following type II collagen sequences are included;

| | |
|---|---|
| GFQGL-Xaa-G-Xaa-Xaa-G-Xaa-Xaa-G | SEQ ID NO: 53 |
| GLQGL-Xaa-G-Xaa-Xaa-G-Xaa-SG | SEQ ID NO: 54 |

Even though a number of the patents mentioned above make reference to the usefulness of type II collagen fragments as markers of cartilage degradation, none of these relates to the detection of the sub-group of type II collagen fragments using an antibody binding specifically to the neo-epitope and the other antibody binding to an isomerised amino acid sequence on the same fragment. Similarly, although the usefulness of measuring isomerised fragments of other proteins such as proteoglycans is disclosed, it has not been appreciated that fragments containing both a proteolytic cleavage neo-epitope and an isomerisation can be measured in a sandwich assay format.

The present invention now provides a method of assay, comprising measuring in a biological sample fragments of a protein that contain an epitope containing an isomerised amino acid residue and a protease generated neo-epitope by binding the neo-epitope with a first immunological binding partner specific for the presence of said neo-epitope and binding the epitope containing said isomerisation with a second immunological binding partner specific for the presence of said isomerisation and detecting the extent of dual binding of said binding partners.

The terms ‘first’ and ‘second’ immunological binding partner above do not of course relate to the order (if there is one) in which the binding partners bind to their targets. One may bind the isomerisation containing sequence of a target peptide first and then bind on the ‘first’ immunological binding partner to the neo-epitope, or one can bind both the isomerisation and the neo-epitope simultaneously, depending on the chosen assay format.

Preferably, said assay is performed as a sandwich assay in which one of said immunological binding partners is immobilised to a solid support, said fragments are bound to said immobilised antibody and the binding of the other of said immunological binding partners to said fragments is detected. Optionally, the assay is performed as a homogeneous sandwich assay.

Preferably, said first immunological binding partner does not specifically bind the intact protein from which said fragments derive and preferably said first immunological binding partner does not specifically bind fragments of said protein containing the amino acid sequence of said neo-epitope extended beyond the protease cleavage site.

Preferably, said neo-epitope is from collagen type II. References to collagen type II herein include specifically reference to human type II collagen.

Optionally, severity of arthritis development in the subject is evaluated by comparing the level of binding measured in said assay with levels previously established in healthy subjects and or in subjects having pathological arthritis activity. For fragments indicative of a different disease, one would of course compare the level of binding with levels relevant to that disease. More preferably, the first immunological binding partner is specific for an epitope defined by one of the following amino acid sequences: . . . GQPGPA [symbol] SEQ ID NO:55; . . . EPGGVG [symbol] SEQ ID NO:56;

[symbol] DQGVPG . . . SEQ ID NO:57; . . . PKGAR [symbol] SEQ ID NO:58; and [symbol] REGSPG . . . SEQ ID NO:59. Preferably, said immunological binding partner does not specifically bind a sequence as defined if continued past the indicated cleavage site.

The invention includes an immunological assay kit comprising a first immunological binding partner specific for an epitope containing an isomerised amino acid residue and a second immunological binding partner specific for a protease generated neo-epitope. Said kit may further include at least one of calibration standards immunoreactive with said binding partners, a wash reagent, a buffer, a secondary immunological binding partner for revealing binding between said first or second immunological binding partner and components of a sample, an enzyme label, an enzyme label substrate, a stopping reagent, and instructions for conducting an assay using said kit.

The biological sample may in particular be a body fluid sample and may be blood, serum, plasma, or urine.

As explained in WO02/095415, isomerisation of certain amino acids occurs naturally over time in proteins of the body, particularly at aspartic acid, asparagines, glutamic acid and glutamine residues, according to the illustrative reaction scheme:

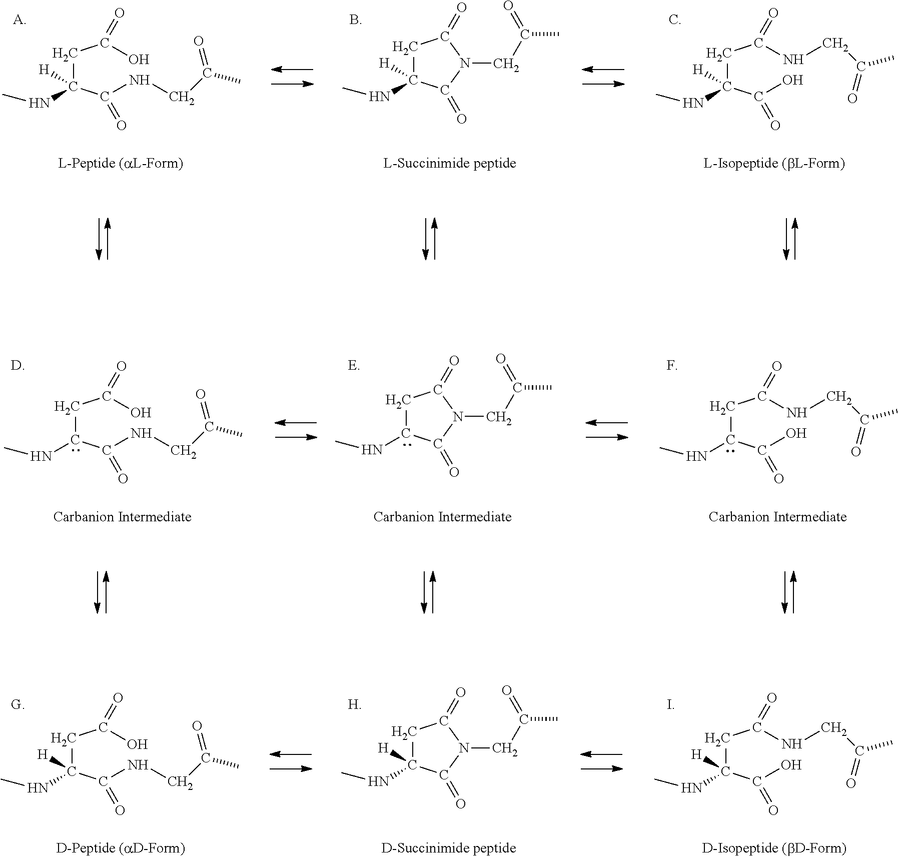

This may give rise to either or both of optical or structural isomerism at the affected residue and either can be recognised in a context specific manner (i.e. dependant on the presence of the appropriate flanking amino acid sequences) or context independent manner (i.e. not dependant on the presence of the appropriate flanking amino acid sequences) by a suitably selected immunological binding partner.

For detecting fragments of Type II collagen, a preferred site of isomerisation to target in an assay is the sequence specifically binds an epitope comprising the sequence -GA(D-β-G)P- SEQ ID NO:60 which occurs in the larger sequence -GSP*GA(D-β-G)PP*GRKK- SEQ ID NO:61 within the sequence of Type II collagen. The invention includes a method of immunoassay for detecting or measuring the rate of breakdown of type II collagen in a subject comprising contacting a body fluid sample from the subject with an immunological binding partner which specifically binds an epitope comprising the sequence -GA(D-β-G)P- SEQ ID NO:60 and detecting or measuring the amount of binding of the immunological binding partner. Such a method may further comprise the use of an immunological binding partner specific for a neo-epitope produced by proteolytic cleavage of Type II collagen as described above.

Assay formats useful in accordance with the present invention include both heterogeneous and homogeneous sandwich assay formats.

Homogeneous formats include the use of two different immunological binding partners bound to respective beads wherein the beads incorporate a detectable proximity signal activated when the beads are brought into proximity by their respective binding partners both binding to sites on a single fragment molecule.

Heterogeneous assay formats include those in which one of said immunological binding partners is immobilised to a solid support, said fragments are bound to said immobilised antibody and the binding of the other of said immunological binding partners to said fragments is detected. Immunological binding partners for use in the present invention include whole antibodies, especially monoclonal antibodies, and antibody fragments with specific binding affinity. These include binding fragments such as Fab or F(ab′)₂.

Assays according to the invention are useful in diagnosis or disease progression monitoring in respect of pathological conditions which lead to the release of fragments detectable in said assays. The disease indicated will depend on the fragments measured. Of especial interest for diagnosis or monitoring of osteoarthritis is collagen II. Other diseases involving destruction of cartilage could also be diagnosed and monitored with the tests according to the present invention. Such disease include rheumatoid arthritis.

Examples of neo-epitopes of proteolytic cleavage of collagen type II include Cathepsin K generated neoepitopes and MMP-generated neoepitopes. Also, included are fragments generated by proteolytic cleavage of type II collagen by aggrecanases, e.g. ADAM-TS4 and ADAM-TS5. The identification of collagen type II sequences carrying Cathepsin K and MMP9 neo-epitopes could be performed as described below. In a similar manner collagen type II sequences carrying other MMP or aggrecanase generated neoepitopes could be identified, or MMP generated neoepitopes could be based on publicly available information on MMP generated peptide products of human articular cartilage (then et al. Arthritis Rheum 2008 58(8):2420-31). To ascertain protein and protease specificity, preferred biomarker neoepitopes of cleavage sites may be selected as follows.

To identify cathepsin K and MMP9 cleavage epitopes in collagen II, human collagen type II (BIOCOL BC-3001) was dissolved in 10 mM acetic acid (400 μl added to 1 mg of collagen type II). Ten μg of procathepsin K (Calbiochem 342001) was activated by addition of 200 μl of 100 mM sodium acetate containing 10 mM DTT and 5 mM EDTA, pH 3.9 for 40 minutes at room temperature. Ten μg of MMP9 (Calbiochem 444231) was activated by addition of 200 μl of 1 mM APMA in DMSO for 2 hours at 37° C. For the Cathepsin K cleavage, 60 μl of collagen type II was added 120 μl of 50 mM sodium acetate, pH 5.5 containing 20 mM L-cystein and 24 μl of activated cathepsin K for 4 hours at 37° C. For the MMP9 cleavage, 60 μl of collagen type II was added 120 μl of 100 mM Tris-HCl, 100 MM sodium chloride, 10 mM calcium chloride, 2 mM zinc chloride, pH 8.0 and 20 μl of MMP9 for 3 days at 37° C. The resulting proteolytic cleavage fragments were characterized by high performance liquid chromatography (HPLC)-tandem mass spectrometry (MS/MS) analysis. The MS/MS spectra were searched against protein databases using Sequest and X! Tandem database search algorithms. The following sequence hits of fragments were found for the Cathepsin K cleaved collagen type II:

| | |
|---|---|
| AQGPPGATGFPGAAGR | SEQ ID NO: 62 |
| ASGDRGPPGPV | SEQ ID NO: 63 |
| ASGDRGPPGPVGPPG | SEQ ID NO: 64 |
| GANGEKGEVGPPGPA | SEQ ID NO: 65 |
| GAPGEDGRPGPPGPQ | SEQ ID NO: 66 |
| GARGAPGERGETGPPGPA | SEQ ID NO: 67 |
| GDRGPPGPV | SEQ ID NO: 68 |
| GERGFPG | SEQ ID NO: 69 |
| GERGFPGER | SEQ ID NO: 70 |
| GESGSPGENGSPGPM | SEQ ID NO: 71 |
| GLPGPPGPPGEGGKPG | SEQ ID NO: 72 |
| GPIGPPGPA | SEQ ID NO: 73 |
| GPPGPPGKPGDDGEAGKPG | SEQ ID NO: 74 |
| GPPGPV | SEQ ID NO: 75 |
| GPPGPVGPA | SEQ ID NO: 76 |
| LPGPPGPPGEGGKPG | SEQ ID NO: 77 |
| NPGPPGPPGPPGPG | SEQ ID NO: 78 |
| PIGPP | SEQ ID NO: 79 |
| REGSPGADGPPGRDGAAGVK | SEQ ID NO: 80 |
| SNGNPGPPGPPGPS | SEQ ID NO: 81 |

Identified fragments were aligned with the sequence for human collagen type II (sp|P02458|CO2A1_HUAN Collagen alpha-1(II) chain), and Cathepsin K cleavage sites were localized as indicated by the arrows.

SEQ ID NO: 82
QMAGGFDEKAGGAQLGVMQGPMGPMGPRGPPGPAGAPGPQGFQGNPGEPG
EPGVSGPMGPR [symbol] GPPGPPGKPGDDGEAGKPG [symbol] KAGERGPPGPQGARGF
PGTPGLPGVKGHRGYPGLDGAKGEAGAPGVK [symbol] GESGSPGENGSPGPM [symbol]
GPRGLPGERGRTGPAGAAGARGNDGQPGPA [symbol] GPPGPV [symbol] GPA [symbol] GGPGF
PGAPGAKGEAGPTGARGPEGAQGPRGEPGTPGSPGPAGASGNPGTDGIPG
AKGSAGAPGIAGAPGFPGPRGPPGPQGATGPLGPKGQTGEPGIAGFKGEQ
GPKGEPGPAGPQGAPGPAGEEGKRGARGEPGGVG [symbol] PIGPP [symbol] GERGAPG
NRGFPGQDGLAGPKGAPGERGPSGLAGPKGANGDPGRPGEPGLPGARGLT
GRPGDAGPQGKVGPS [symbol] GAPGEDGRPGPPGPQ [symbol] GARGQPGVMGFPGPKG
ANGEPGKAGEKGLPGAPGLRGLPGKDGETGAAGPPGPAGPAGERGEQGAP
GPSGFQ [symbol] G [symbol] LPGPPGPPGEGGKPG [symbol] DQGVPGEAGAPGLVGPR [symbol] GER
GFPG [symbol] ER [symbol] GSPGAQGLQGPRGLPGTPGTDGPKGASGPAGPPGAQGPPG
LQGMPGERGAAGIAGPKGDRGDVGEKGPEGAPGKDGGRGLT [symbol] GPIGPPG
PA [symbol] GANGEKGEVGPPGPA [symbol] GSA [symbol] GARGAPGERGETGPPGPA [symbol] GFAG
PPGADGQPGAKGEQGEAGQKGDAGAPGPQGPSGAPGPQGPTGVTGPKGARG [symbol]
AQGPPGATGFPGAAGR [symbol] VGPPG [symbol] SNGNPGPPGPPGPS [symbol] GKDGPKG
ARGDSGPPGRAGEPGLQGPAGPPGEKGEPGDDGPSGAEGPPGPQGLAGQRG
IVGLPGQRGERGFPGLPGPSGEPGKQGAPG [symbol] AS [symbol] GDRGPPGPV [symbol] GPP
G [symbol] LIGPAGEPG [symbol] REGSPGADGPPGRDGAAGVK [symbol] GDRGETGAVGAPGA
PGPPGSPGPAGPTGKQGDRGEAGAQGPMGPSGPAGARGIQGPQGPRGDKGE
AGEPGERGLKGHRGFTGLQGLPGPPGPSGDQGASGPAGPSGPRGPPGPVGP
SGKDGANGIPGPIGPPGPRGRSGETGPAGPPG [symbol] NPGPPGPPGPPGPG [symbol]
IDMSAFAGLGPREKGPDPLQYMRA

The following sequence hits of fragments were found for the MMP9 cleaved collagen type II:

SEQ ID NO: 83
AAGARGNDGQPGPAGPPGPVGPA
SEQ ID NO: 84
AQGPRGEPGTPGSPGPAG
SEQ ID NO: 85
ARGAPGERGETGPPGPAG
SEQ ID NO: 86
ASGDRGPPGPV
SEQ ID NO: 87
ASGDRGPPGPVG
SEQ ID NO: 88
ATGPLGPKG
SEQ ID NO: 89
DRGPPGPVGPPG
SEQ ID NO: 90
ERGAPGNRGFPGQDGLAGPKGAPGERGPSG
SEQ ID NO: 91
FQGLPGPPGPPGEGGKPGDQGVPGEAGAPGLVGPR
SEQ ID NO: 92
FQGLPGPPGPPGEGGKPGDQGVPGEAGAPGLVGPRG
SEQ ID NO: 93
FTGLQGLPGPPGPSG
SEQ ID NO: 94
FTGLQGLPGPPGPSGDQGASGPAGPSGPRGPPGPVGPSG
SEQ ID NO: 95
GANGEKGEVGPPGPA
SEQ ID NO: 96
GAPGEDGRPGPPGPQ
SEQ ID NO: 97
GAPGEDGRPGPPGPQG
SEQ ID NO: 98
GAPGERGETGPPGPA
SEQ ID NO: 99
GESGSPGENGSPGPM
SEQ ID NO: 100
GPIGPPGPA
SEQ ID NO: 75
GPPGPV
SEQ ID NO: 101
GPPGPVGPPG
SEQ ID NO: 102
GPRGPPGPAGAPGPQG
SEQ ID NO: 103
GVMQGPMGPMGPRGPPGPAGAPGPQG
SEQ ID NO: 104
IVGLPGQRGERGFPGLPGPSGEPGK
SEQ ID NO: 105
KQGDRGEAGAQGPMGPSGPAG
SEQ ID NO: 106
KVGPSGAPGEDGRPGPPGPQG
SEQ ID NO: 107
LPGKDGETGAAGPPGPAGPAG
SEQ ID NO: 108
LPGKDGETGAAGPPGPAGPAGERGEQGAPGPSG
SEQ ID NO: 109
LQGLPGPPGPSGDQGASGPAGPSGPRGPPGPVGPSG
SEQ ID NO: 110
LTGPAGEPGREGSPGAD
SEQ ID NO: 111
LTGPAGEPGREGSPGADGPPGRDGAAG
SEQ ID NO: 112
LTGPIGPPGPAG
SEQ ID NO: 113
LTGRPGDAGPQGKVGPSGAPGEDGRPGPPGPQG
SEQ ID NO: 114
QGPMGPMGPRGPPGPAGAPGPQG
SEQ ID NO: 115
QGPRGLPGTPGTDGPKGASGPAGPPGAQGPP
SEQ ID NO: 116
RSGETGPAGPPGNPGPPGPPGPPGPGID
SEQ ID NO: 117
RVGPPGSNGNPGPPGPPGPSG
SEQ ID NO: 118
SNGNPGPPGPPGPS
SEQ ID NO: 119
SPGPMGPRG
SEQ ID NO: 120
VKGESGSPGENGSPGPMGPRG

Identified MMP9-generated fragments were aligned with the sequence for human collagen type II (sp|P02458|CO2A1_HUAN Collagen alpha-1(II) chain), and cleavage sites were localized as indicated by the stars.

SEQ ID NO: 82
QMAGGFDEKAGGAQL*GVM*QGPMGPM*GPRGPPGPAGAPGPQG*FQGNP
GEPGEPGVSGPMGPRGPPGPPGKPGDDGEAGKPGKAGERGPPGPQGARGF
PGTPGLPGVKGHRGYPGLDGAKGEAGAPG*VK*GESGSPGENG*SPGPM*
GPRG*LPGERGRTGPAG*AAGARGNDGQPGPAGPPGPVGPA*GGPGFPGA
PGAKGEAGPTGARGPEG*AQGPRGEPGITGSPGPAG*ASGNPGTDGIPGA
KGSAGAPGIAGAPGFPGPRGPPGPQG*ATGPLGPKG*QTGEPGTAGFKGE
QGPKGEPGPAGPQGAPGPAGEEGKRGARGEPGGVGPIGPPG*ERGAPGNR
GFPGQDGLAGPKGAPGERGPSG*LAGPKGANGDPGRPGEPGLPGARG*LT
GRPGDAGPQG*KVGPS*GAPGEDGRPGPPGPQ*G*ARGQPGVMGFPGPKG
ANGEPGKAGEKGLPGAPGLRG*LPGKDGETGAAGPPGPAGPAG*ERGEQG
APGPSG*FQGLPGPPGPPGEGGKPGDQGVPGEAGAPGLVGPR*G*ERGFP
GERGSPGAQGL*QGPRGLPGTPGTDGPKGASGPAGPPGAQGPP*GLQGMP
GERGAAGIAGPKGDRGDVGEKGPEGAPGKDGGRG*LT*GPIGPPGPA*G*
ANGEKGEVGPPGPA*GSAG*AR*GAPGERGETGPPGPA*G*FAGPPGADG
QPGAKGEQGEAGQKGDAGAPGPQGPSGAPGPQGPTGVTGPKGARGAQGPP
GATGFPGAAG*RVGPPG*SNGNPGPPGPPGPS*G*KDGPKGARGDSGPPG
RAGEPGLQGPAGPPGEKGEPGDDGPSGAEGPPGPQGLAGQRG*IVGLPGQ
RGERGFPGLPGPSGEPGK*QGAPG*ASG*DR*GPPGPV*G*PPG*LIGPA
GEPGREGSPGAD*GPPGRDGAAG*VKGDRGETGAVGAPGAPGPPGSPGPA
GPTG*KQGDRGEAGAQGPMGPSGPAG*ARGIQGPQGPRGDKGEAGEPGER
GLKGHRG*FTG*LQGLPGPPGPSG*DQGASGPAGPSGPRGPPGPVGPSG*
KDGANGIPGPIGPPGPRG*RSGETGPAGPPGNPGPPGPPGPPGPGID*MS
AFAGLGPREKGPDPLQYMRA

Preferred biomarker neoepitopes were selected based on protein specificity. The protein specificity of cleavage sites were assessed by identity search of 6 amino-terminal or 6 carboxy-terminal residues on either site of the cleavage site. The public available programme “Pattinprot” was used in the search of the UNIPROT/SWISSPROT databank. Neoepitopes that could be non-specific for collagen type II were deselected, that is deselected if a similar six-residue amino acid sequence was present in human collagen type I or other human proteins of major abundance. The preferred biomarker neoepitopes of Cathepsin K cleavage sites (arrows) and/or MMP cleavage sites (stars) are indicated by underlined sequences:

SEQ ID NO: 82
QMAGGFDEKAGGAQLGVMQGPMGPMGPRGPPGPAGAPGPQGFQGNPGEPG
EPGVSGPMGPRGPPGPPGKPGDDGEAGKPGKAGERGPPGPQGARGFPGTP
GLPGVKGHRGYPGLDGAKGEAGAPGVKGESGSPGENGSPGPMGPRGLPGE
RGRTGPAGAAGARGNDGQPGPA [symbol] GPPGPVGPAGGPGFPGAPGAKGEAGPT
GARGPEGAQGPRGEPGTPGSPGPAGASGNPGTDGIPGAKGSAGAPGIAGA
PGFPGPRGPPGPQGATGPLGPKGQTGEPGIAGFKGEQGPKGEPGPAGPQG
APGPAGEEGKRGARGEPGGVG [symbol] PIGPPGERGAPGNRGFPGQDGLAGPKGA
PGERGPSG*LAGPKGANGDPGRPGEPGLPGARGLTGRPGDAGPQGKVGPS
GAPGEDGRPGPPGPQGARGQPGVMGFPGPKGANGEPGKAGEKGLPGAPGL
RGLPGKDGETGAAGPPGPAGPAGERGEQGAPGPSGFQGLPGPPGPPGEGG
KPG [symbol] DQGVPGEAGAPGLVGPRGERGFPGERGSPGAQGLQGPRGLPGTPGT
DGPKGASGPAGPPGAQGPPGLQGMPGERGAAGIAGPKGDRGDVGEKGPEG
APGKDGGRGLTGPIGPPGPAGANGEKGEVGPPGPAGSAGARGAPGERGET
GPPGPAGFAGPPGADGQPGAKGEQGEAGQKGDAGAPGPQGPSGAPGPQGP
TGVTGPKGARG [symbol] AQGPPGATGFPGAAGRVGPPGSNGNPGPPGPPGPSGKD
GPKGARGDSGPPGRAGEPGLQGPAGPPGEKGEPGDDGPSGAEGPPGPQGL
AGQRGIVGLPGQRGERGFPGLPGPSGEPGKQGAPGASGDRGPPGPVGPPG
* [symbol] LTGPAGEPG [symbol] REGSPGADGPPGRDGAAG*VKGDRGETGAVGAPGAPGP
PGSPGPAGPTGKQGDRGEAGAQGPMGPSGPAGARGIQGPQGPRGDKGEAG
EPGERGLKGHRGFTGLQGLPGPPGPSGDQGASGPAGPSGPRGPPGPVGPS
GKDGANGTPGPTGPPGPRGRSGETGPAGPPGNPGPPGPPGPPGPGTDMSA
FAGLGPREKGPDPLQYMRA

An additional requirement for preferred biomarker fragments is localization of cleavage sites in proximity of a DG beta-isomerized sequence. Collagen II neo-epitopes of particular interest therefore include the Cathepsin K generated N-terminal neoepitope site ↓REGSPGADGPP SEQ ID NO:121, and examples of MMP generated neopitope sites include the sequence ↓LTGPAGEPGREGSPGADGPPGR SEQ ID NO:122 (N-terminal neoepitope) and the sequence GSPGADGPPGRDGAAG↓ SEQ ID NO:123 (C-terminal neoepitope).

The invention will be further described and illustrated by the following examples making reference to the accompanying drawings, in which:

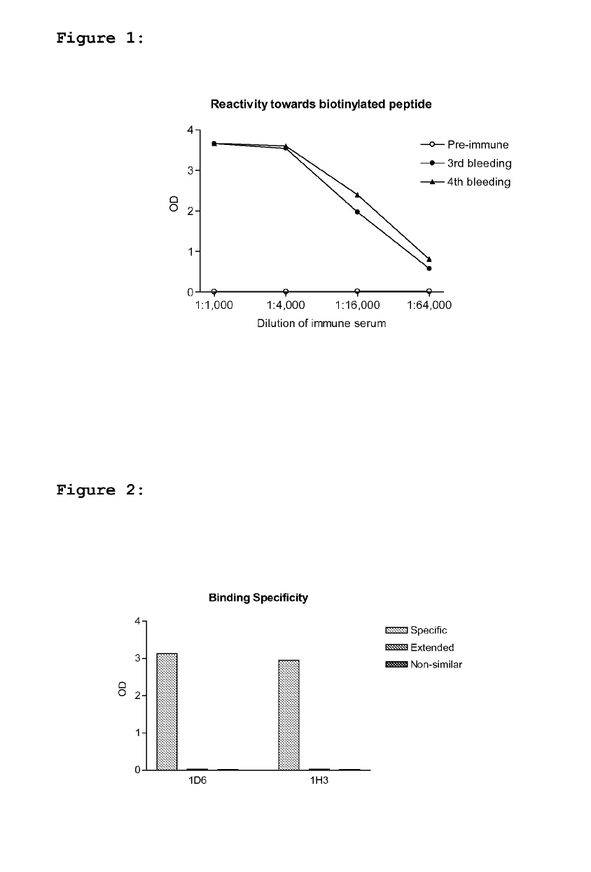

FIG. 1 shows antibody binding in sera studied in Example 1;

FIG. 2 shows monoclonal antibody binding observed in Example 1; and

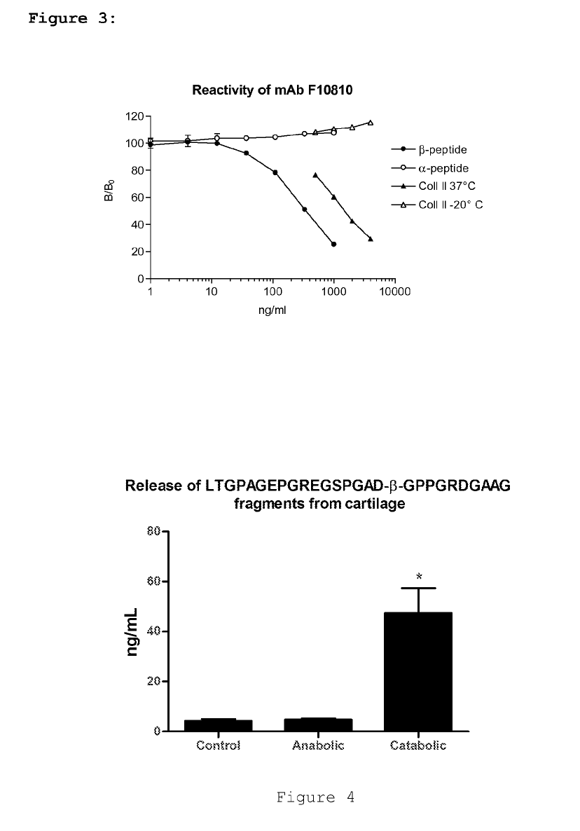

FIG. 3 shows monoclonal antibody binding observed in Example 2.

FIG. 4 shows monoclonal antibody binding observed in Example 4.

### EXAMPLE 1

### Generation of Monoclonal Antibodies Recognising a N-Terminal Neoepitope

Synthetic peptides were prepared by standard techniques. To increase immunogenicity, the peptide (LTGPAGGGGCSEQ ID NO:124) was coupled at the C-terminus to the carrier protein KLH using site-directed coupling technology via the cysteine. Before immunisation, the immunogen was mixed 1:1 with Freund's Incomplete Adjuvant and the mixture was injected s.c. in Balb/c mice. The immunisation was repeated every 2 weeks for two months (four immunisations) and then continued with 4 weeks between each immunisation. Blood was obtained from the mice before immunisation initiated and one week after each immunization. The immune response was evaluated by testing the binding reactivity of mouse immune sera towards the C-terminal biotinylated synthetic peptide (LTGPAGEPGK-Biotin) (SEQ ID NO:125). The test for binding reactivity of mouse immune sera was based on binding of the immune serum to the biotinylated synthetic peptide that was bound to the surface of a streptavidin-coated microtitre plate. After incubation and washing the bound antibody was demonstrated by incubation with anti-mouse IgG conjugated to horseradish peroxidase, washing and addition of the chromogen TMB (FIG. 1).

Subsequent to attaining sufficient immune sera titers in the above mentioned screening test, the selected mice were rested for at least 4 weeks, and boosted i.p. with immunogen without adjuvants. Three days later, the spleen was removed and used for fusion with myeloma cells using standard techniques. Antibodies from growing hybridomas were evaluated by their binding reactivity to the biotinylated synthetic peptide in the assay as described above. Additionally the cleavage specificity of the antibodies was demonstrated by minimum binding reactivity towards a one-residue extended coater SEQ ID NO:126 (GLTGPAGEPGK-Biotin) as well as no binding towards a non-similar coater SEQ ID NO:127 (Biotin-KGATGPLGPK)(FIG. 2).

### EXAMPLE 2

### Generation of Monoclonal Antibodies Recognising a P-Isomerized Site

A beta-isomerised site of collagen type II is ‘SP*GA(D-β-G)PP*GR’ SEQ ID NO:128 as described in patent WO 02/095415 A2 (P* indicates hydroxyproline). ‘D-β-G’ indicates aspartic acid bound to glycine by the beta carboxylic acid of aspartic acid.

Synthetic peptides were prepared by standard techniques. To increase immunogenicity, the peptide (CSP*GA(D-β-G)PP*GR) SEQ ID NO:129 was coupled at the N-terminus to the carrier protein KLH using site-directed coupling technology via the cysteine. An immunization schedule similar to that of Example 1 was used. The immune response was evaluated by testing the binding reactivity of mouse immune sera towards the C-terminal biotinylated synthetic peptide SEQ ID NO:61 (GSP*GA(D-β-G)PP*GRKK-Biotin). The test for binding reactivity of mouse immune sera was based on binding of the immune serum to the biotinylated synthetic peptide that was bound to the surface of a streptavidin-coated microtitre plate. After incubation and washing the bound antibody was demonstrated by incubation with europium-labelled anti-mouse IgG, washing and reading of the time-resolved fluorescence signal.

Subsequent to attaining sufficient immune sera titers, mice were rested, boosted, and used for production of hybridomas as described in Example 1. Antibodies from growing hybridomas were evaluated by their binding reactivity to the biotinylated synthetic peptide in the assay as described above. The isoform specificity of the antibodies was demonstrated by maximal displacement of the antibody binding by the beta-form of the synthetic peptide, GSP*GA(D-β-G)PR*GR SEQ ID NO:128, and minimum displacement by the alpha-form of the synthetic peptide, GSP*GADGPP*GR SEQ ID NO:130, as well as higher degree of displacement by in-vitro beta-isomerised collagen type II (collagen incubated at 37° C. for 3 months) in comparison with same material stored at −20° C. (FIG. 3).

### EXAMPLE 3

### Detection of Collagen Type II Fragments Carrying Both a MMP-Generated Neo-Epitope and Isomerisation

One monoclonal antibody binding to the N-terminus of the amino acid sequence LTGPAGGGGC SEQ ID NO:124 as described above was biotinylated according to standard procedures and used as capture antibody in a sandwich ELISA. The detector antibody, binding to the isomerised amino acid sequence CSP*GA(D-β-G)PP*GR SEQ ID NO:129 as described above, was labelled with horseradish peroxidase according to standard techniques. Using these two reagents and a microtitre plate coated with streptavidin, the antibodies was pre-diluted in a 10 mM phosphate buffered solution (PBS) with bovine serum albumin (1%) and Tween 20 (0.1%) in a total volume of 100 μL and was subsequently incubated in the microtitre plate with 50 μL of human serum sample. A synthetic peptide with the amino acid sequence LTGPAGEPG [symbol] REGSPGAD-β-GPPGRDGAAGVK SEQ ID NO:131 containing both the neo-epitope and the isomerised amino acid sequence was used as calibrator. After one hour of incubation at 20° C. the wells were washed in PBS with 0.1% Tween 20 and subsequently the colour reaction was initiated by addition of 100 μL of chromogen (TMB). After 15 minutes the reaction was stopped by addition of 0.18M sulphuric acid.

Measurement of 20 human serum samples originating from patients with OA and 20 samples from healthy subjects in the ELISA described above demonstrated will be tested and an increase of >50% (p<0.05) in the concentration of the collagen type II fragments in the diseased subjects is expected.

### EXAMPLE 4

### Detection of Collagen Type II Fragments Carrying Both a MMP-Generated Neo-Epitope and Isomerisation in Culture Supernatants of Human Cartilage Explants

One monoclonal antibody binding to the N-terminus of the amino acid sequence LTGPAGGGGC SEQ ID NO:124 as described above was biotinylated according to standard procedures and used as capture antibody in a sandwich ELISA. The detector antibody, binding to the isomerised amino acid sequence GSP*GA(D-β-G)PP*GR SEQ ID NO:128 as described above, was labelled with horseradish peroxidase according to standard techniques. Using these two reagents and a microtitre plate coated with streptavidin, the antibodies were pre-diluted in a 10 mM phosphate buffered solution (PBS) with bovine serum albumin (1%) and Tween 20 (0.1%) in a total volume of 100 μL and subsequently incubated in the microtitre plate with 50 μL of culture supernatants. A synthetic peptide with the amino acid sequence LTGPAGEPG [symbol] REGSPGAD-β-GPPGRDGAAGVK SEQ ID NO:131 containing both the neo-epitope and the isomerised amino acid sequence was used as calibrator. After one hour of incubation at 20° C. the wells were washed in PBS with 0.1% Tween 20 and subsequently the colour reaction was initiated by addition of 100 μL of TMB chromogen (3-3′,5,5′-tetramethylbenzidine). After 15 minutes the reaction was stopped by addition of 0.18M sulphuric acid.

The fragment was detected in culture supernatants of human cartilage explants from osteoarthritic patients, after treatment of the tissue with catabolic or anabolic cytokines. The fragment release was significantly elevated in the catabolic treated cartilage cultures (FIG. 4). This demonstrates that the fragment is present and released from human cartilage and that it is a marker of cartilage turnover.

### EXAMPLE 5

### Generation of Monoclonal Antibodies Recognising N- or C-Terminal Cathepsin K Neoepitopes

The following further demonstrates that monoclonal antibodies can be generated against cathepsin K mediated collagen type II neo-epitopes. To increase immunogenicity, the peptides EAGKPG [symbol] (SEQ ID NO:132—NB76), GQPGPA [symbol] (SEQ ID NO:55—NB77), EPGGVG [symbol] (SEQ ID NO:56—NB78), [symbol] DQGVPG (SEQ ID NO:57—NB79), PKGARG [symbol] (SEQ ID NO:58—NB80) and [symbol] REGSPG (SEQ ID NO:59—NB81) were coupled at the N- or C-terminus as appropriate (i.e. were coupled at the end opposite the marked cleavage site) to the carrier protein KLH using site-directed coupling technology via the cysteine, which is added to the synthetic peptide. Before immunisation, the immunogen was mixed 1:1 with Freund's Incomplete Adjuvant and the mixture was injected s.c. in Balb/c mice. The immunisation was repeated every 2 weeks for two months (four immunisations) and then continued with 4 weeks between each immunisation. Blood was obtained from the mice before immunisation was initiated and one week after each immunization. The immune response was evaluated by testing the binding reactivity of mouse immune sera towards the C-terminal biotinylated synthetic peptide corresponding to each of the selected sequences (e.g. REGSPGGADAP-Biotin SEQ ID NO:133). The test for binding reactivity of mouse immune sera was based on binding of the immune serum to the biotinylated synthetic peptide that was bound to the surface of a streptavidin-coated micro-titre plate. After incubation and washing the bound antibody was demonstrated by incubation with anti-mouse IgG conjugated to horseradish peroxidase, washing and addition of the chromogen TMB. Subsequent to attaining sufficient immune sera titers in the above mentioned screening test, the selected mice were rested for at least 4 weeks, and boosted i.p. with immunogen without adjuvants. Three days later, the spleen was removed and used for fusion with myeloma cells using standard techniques. Antibodies from growing hybridomas were evaluated by their binding reactivity to the biotinylated synthetic peptide in the assay as described above.

An assay combining use of the 13 isomerised epitope binding antibody of Example 2 and the antibody described above to bind collagen II fragments exhibiting both epitopes can be constructed using the principles described herein. In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

Zhen E Y, Brittain I J, Laska D A, Mitchell P G, Sumer E U, Karsdal M A, Duffin KL. Characterization of metalloprotease cleavage products of human articular cartilage. Arthritis Rheum. 2008 August; 58(8):2420-31
Kafienah W, Brömme D, Buttle D J, Croucher L J, Hollander A P. Human cathepsin K cleaves native type I and II collagens at the N-terminal end of the triple helix. Biochem J. 1998 May 1; 331 (Pt 3):727-32
Dejica V M, Mort J S, Layerty S, Percival M D, Antoniou J, Zukor D J, Poole A R. Cleavage of type II collagen by cathepsin K in human osteoarthritic cartilage. Am J. Pathol. 2008 July; 173(1):161-9. Epub 2008 May 29
Dean, D. D., Martel-Pelletier, J., Pelletier, J. P., Howell, D. S., and Woessner, J. F., Jr. 1989. Evidence for metalloproteinase and metalloproteinase inhibitor imbalance in human osteoarthritic cartilage. J Clin Invest 84:678-685.
Reboul, P., Pelletier, J. P., Tardif, G., Cloutier, J. M., and Martel-Pelletier, J. 1996. The new collagenase, collagenase-3, is expressed and synthesized by human chondrocytes but not by synoviocytes. A role in osteoarthritis. J Clin Invest 97:2011-2019.
Hui, W., Rowan, A. D., Richards, C. D., and Cawston, T. E. 2003. Oncostatin M in combination with tumor necrosis factor alpha induces cartilage damage and matrix metalloproteinase expression in vitro and in vivo. Arthritis Rheum 48:3404-3418.
Schaller, S., Henriksen, K., Hoegh-Andersen, P., Sondergaard, B. C., Sumer, E. U., Tanko, L. B., Qvist, P., and Karsdal, M. A. 2005. In vitro, ex vivo, and in vivo methodological approaches for studying therapeutic targets of osteoporosis and degenerative joint diseases: how biomarkers can assist? Assay. Drug Dev. Technol. 3:553-580.
Sumer, E. U., Schaller, S., Sondergaard, B. C., Tanko, L. B., and Qvist, P. 2006. Application of biomarkers in the clinical development of new drugs for chondroprotection in destructive joint diseases: a review. Biomarkers 11:485-506.
Christgau, S., Garnero, P., Fledelius, C., Moniz, C., Ensig, M., Gineyts, E., Rosenquist, C., and Qvist, P. 2001. Collagen type II C-telopeptide fragments as an index of cartilage degradation. Bone 29:209-215.
Mouritzen, U., Christgau, S., Lehmann, H. J., Tanko, L. B., and Christiansen, C. 2003. Cartilage turnover assessed with a newly developed assay measuring collagen type II degradation products: influence of age, sex, menopause, hormone replacement therapy, and body mass index. Ann. Rheum Dis 62:332-336.
Reijman, M., Hazes, J. M., Bierma-Zeinstra, S. M., Koes, B. W., Christgau, S., Christiansen, C., Uitterlinden, A. G., and Pols, H. A. 2004. A new marker for osteoarthritis: cross-sectional and longitudinal approach. Arthritis Rheum 50:2471-2478.
Woolf A D, Pfleger B: Burden of major musculoskeletal conditions. Bull World Health Organ 2003; 81:646-56
Lawrence R C, Helmick C G, Arnett F C et al. Estimates of the prevalence of arthritis and selected musculoskeletal disorders in the United States. Arthritis Rheum 1998; 41:778-99.
Pritzker K P, Gay S, Jimenez S A, Ostergaard K, Pelletier J P, Revell P A, Salter D, van den Berg W B. Osteoarthritis cartilage histopathology: grading and staging. Osteoarthritis Cartilage 2006; 14:13-29.
Birmingham J D, Vilim V, Kraus V B. Collagen biomarkers for arthritis applications. Biomarker Insights 2006; 2: 61-76.
Fraser A, Fearon U, Billinghurst R C, Ionescu M, Reece R, Barwick T, Emery P, Poole A R, Veale D J. Turnover of type II collagen and aggrecan in cartilage matrix at the onset of inflammatory arthritis in humans: relationship to mediators of systemic and local inflammation. Arthritis Rheum. 2003 November; 48(11):3085-95.
Billinghurst R C, Dahlberg L, Ionescu M, Reiner A, Bourne R, Rorabeck C, Mitchell P, Hambor J, Diekmann O, Tschesche H, Chen J, Van Wart H, Poole A R.
Enhanced cleavage of type II collagen by collagenases in osteoarthritic articular cartilage. J Clin Invest. 1997 Apr. 1; 99(7):1534-45.
Poole A R, Ionescu M, Fitzcharles M A, Billinghurst R C. The assessment of cartilage degradation in vivo: development of an immunoassay for the measurement in body fluids of type II collagen cleaved by collagenases. J Immunol Methods. 2004 November; 294(1-2):145-53.
Otterness I G, Downs J T, Lane C, Bliven M L, Stukenbrok H, Scampoli D N, Milici A J, Mezes P S. Detection of collagenase-induced damage of collagen by 9A4, a monoclonal C-terminal neoepitope antibody. Matrix Biol. 1999 August; 18(4):331-41.
Downs J T, Lane C L, Nestor N B, McLellan T J, Kelly M A, Karam G A, Mezes P S, Pelletier J P, Otterness I G. Analysis of collagenase-cleavage of type II collagen using a neoepitope ELISA. J Immunol Methods. 2001 Jan. 1; 247(1-2):25-34.

1. A method of assay, comprising measuring in a biological sample fragments of a protein that contain an epitope containing an isomerised amino acid residue and a protease generated neo-epitope by binding the neo-epitope with a first immunological binding partner specific for the presence of said neo-epitope and binding the epitope containing said isomerisation with a second immunological binding partner specific for the presence of said isomerisation and detecting the extent of dual binding of said binding partners.

2. A method as claimed in claim 1, wherein said assay is performed as a sandwich assay in which one of said immunological binding partners is immobilised to a solid support, said fragments are bound to said immobilised antibody and the binding of the other of said immunological binding partners to said fragments is detected.

3. A method as claimed in claim 1, wherein the assay is performed as a homogeneous sandwich assay.

4. A method as claimed in claim 1, wherein said first immunological binding partner does not specifically bind the intact protein from which said fragments derive.

5. A method as claimed in claim 4, wherein said first immunological binding partner does not specifically bind fragments of said protein containing the amino acid sequence of said neo-epitope extended beyond the protease cleavage site.

6. A method as claimed in claim 1, wherein said neo-epitope is from collagen type II.

7. A method as claimed in claim 6, wherein the first immunological binding partner is specific for an epitope defined by one of the following amino acid sequences: . . . GQPGPA [symbol] SEQ ID NO:55; . . . EPGGVG [symbol] SEQ ID NO:56; [symbol] DQGVPG . . . SEQ ID NO:57; . . . . PKGARG [symbol] SEQ ID NO:58; and [symbol] REGSPG . . . SEQ ID NO:59.

8. A method as claimed in claim 7, wherein said immunological binding partner does not specifically bind a sequence as defined in claim 7 if continued past the indicated cleavage site.

9. A method as claimed in claim 6, wherein said second immunological binding partner specifically binds an epitope comprising the sequence -GA(D-β-G)P- SEQ ID NO:60.

10. A method as claimed in claim 9, wherein said second immunological binding partner specifically binds peptide fragments comprising the sequence -GSP*GA(D-β-G)PP*GRKK- SEQ ID NO:61.

11. A immunological assay kit comprising a first immunological binding partner specific for a protease generated neo-epitope and a second immunological binding partner specific for an epitope containing an isomerised amino acid residue.

12. A kit as claimed in claim 11, wherein said first immunological binding partner is specific for an epitope defined by one of the following amino acid sequences: . . . GQPGPA [symbol] SEQ ID NO:55; . . . EPGGVG [symbol] SEQ ID NO:56; [symbol] DQGVPG . . . SEQ ID NO:57; . . . . PKGARG [symbol] SEQ ID NO:58; and [symbol] REGSPG . . . SEQ ID NO:59.

13. A kit as claimed in claim 11, wherein said second immunological binding partner specifically binds an epitope comprising the sequence -GA(D-β-G)P-.

14. A method of immunoassay for detecting or measuring the rate of breakdown of type II collagen in a subject comprising contacting a body fluid sample from the subject with an immunological binding partner which specifically binds an epitope comprising the sequence -GA(D-β-G)P- and detecting or measuring the amount of binding of the immunological binding partner.
